(19) United States
(12) Patent Application Publication
(10) Pub. No.: US 2010/0023032 A1
(43) Pub. Date: Jan. 28, 2010

(54) PROSTHESIS FOR ANASTOMOSIS

(76) Inventor: Luiz Gonzaga Granja Filho, Recife/PE (BR)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 12/303,543

(22) PCT Filed: Jun. 6, 2007
(86) PCT No.: PCT/BR07/00143

§ 371 Date: Jun. 18, 2009

(30) Foreign Application Priority Data
Jun. 6, 2006 (BR) PI 0602379-7

(51) Int. Cl. A61B 17/08

(52) U.S. Cl. 606/153

(57) ABSTRACT

Prosthetic devices are provided that are used for anastomosis on end-to-side, end-to-end, and side-to-side without clamping and sutureless, or with quick clamping and sutureless. The prosthetic device can have an external flange, rigid or flexible, whether having elastic memory or not, and intraluminal parts having small or none rigid part, compact, and prolonged by metallic wire that makes possible anastomosis without clamping and sutureless. Prosthesis may also have several dimensions and formats in order to accommodate sizes, number and several types of implants in a simultaneous way.

### FIELD OF INVENTION

The present invention generally refers to anastomotic devices and, more specifically to a prosthetic device being able or not to have an external flange, rigid or flexible, and intraluminal parts having small or none rigid part, sustained by metallic wire or any other, resistant and having elastic memory in a way of forming shafts that make possible anastomosis without clamping and sutureless, or quick clamping and sutureless wherein at least one vascular implant, or any other, is inserted in the prosthesis lumen and it is reversed by jacketing to cover part of the prosthesis that will stay inside the everted implant (vein, artery or any tissue) and it is affixed in the rigid intraluminal part, through a circular stitch and/or on the flange of the prosthesis, through simple separated stitches. The solid flange of the prosthesis may have a plurality of spaced openings in its outer edge or chamfers in perimeter, allowing the prosthesis to be sutured on tissue, vein, artery or any other organ out of anastomosis, thus eliminating one of the main causes of obstruction on anastomosis that is the introduction of strange bodies inside lumen as well as anastomosis to be done without clamping that is the main responsible factor for tromboembolism, lacerations of friable walls and ischemia of organs due to clamping. It also permits wide anastomosis with no risk of implants elbowing, single or multiple, at a single time.

### DESCRIPTION OF THE PRIOR ART

A prior art presents several trials provide solutions for anastomotic devices projected to correct vascular abnormalities, which present the following typical features:

The North-American patent U.S. Pat. No. 3,254,650, of Jun. 7, 1966, describes a method and devices to execute anastomosis procedures by applying with adhesive two separated connectors in a body member and removing this body member portion contained among the connectors, joining the said connection devices for joining the remaining portions of the body member.

The U.S. Pat. No. 3,265,069, of Aug. 9, 1966, describes devices or instruments for use by surgeons in reunion of body ducts, which in the course of operations were separated. The instruments comprise a pair of elongated similar elements and articulatedly connected, in an intermediary manner, and with an support for finger retention in a distal end, comprising a generally cylindrical shape with a cylindrical channel that passes through it in the other distal end, in order to receive tubular body ducts kept by the instrument while the body ducts are reconnected.

U.S. Pat. No. 3,774,615, of Nov. 27, 1973, describes a device to connect the end of interrupted tubular organs without sewing, comprising a connecting ring on which the end of the interrupted organ are pulled, the ring is preferably locked up by a fixation resource. The ring and fixation resource are made of inert material, and preferably a hydrophile gel that can be dilated until its equilibrium or can be a hydrogel incompletely dilated, which is submitted to additional dilatation where it is applied. The connecting ring can be supplied with a groove and can be placed in a ring shaped fixation resource and kept there joining it to the fixation resource in the groove or simply kept by a screw. Two connection rings can also be used and kept joined by a coupling member.

The document U.S. Pat. No. 4,366,819, of Jan. 4, 1983, describes an anastomotic joint for surgery with a graft of coronary artery deviance comprising a mounting of four elements including a cylindrical tube with at least one locking indentation of ring flange in one influx end and a plurality of grooves of locking ring in a flow end; a ring flange with a central opening and a plurality of long and short spigots, the long spigots are engaged in the locking indentation, with a graft engaged among them; a fixation ring with a central opening and a plurality of spigots positioned around the opening; and a locking ring with a opening with a plurality of locking ring edges for engaging with the locking ring grooves. In surgical implants, an aortic wall with a hole engages between the ring flange and the fixation ring and is kept in this position by spigots of the fixation ring, and the four elements engage together forming an integral anastomotic joint. A first alternate modality includes an anastomotic joint of three elements with a combination of fixation ring and locking ring. A second alternate modality includes an anastomotic joint of four elements with a slightly jolted end in an influx end, exposing the graft material in the anastomotic “ostium”.

Other prior arts are equally mentioned, base don some information of “The Cardiothoracic Surgery Network”. The “Simmetry Aortic Connector System”, developed by St. Jude Medical, is a connector made with nitinol, selected by vein diameter with an adventitia removed to allow adjust of the connector and to prevent its displacement by the blood current. Then, the device may make an angle of 90° with the aorta. Among the disadvantages, there is the fact that it can be used only in extreme cases due to the difficult usage of this technique; it did not obtain a satisfactory result in many surgeries and it is being drowned out of market by the manufacturer; it is not applicable in calcified aorta; presents suture; presents contact with blood flow (foreign body); it does not widen the anastomosis area (restrictive anastomosis); performs only one anastomosis at a time; it is a product restrict to end-to-side anastomosis; a great mobilization of the venous graft occurs, damaging it, and can eventually form thrombus; there is a risk of perforation of the posterior wall of aorta; and the adventitia is removed (most resistant vascular layer).

Other known device is the PAS-Port™ System, a device used in 3 steps, and the vein wall is mounted over the device and is manually reversed on it, by tool and adapted to aorta with an angle of 90°. The method alerts that the surgeon shall select with due care the point of aorta and the vein size. The device is made of stainless steel and is available in only one size that allows the use of veins with external diameter of 4 to 6 mm, aorta with an internal diameter of 18 mm. It is available in only one size, limiting its applicability. As disadvantages of this prior art, the device has contact with blood flow (foreign body); it does not widen the anastomosis area (restrictive anastomosis); it uses veins with external diameter of 4 to 6 mm and aorta with an internal diameter of 18 mm; it does not perform multiple nor visceral anastomosis; it performs just only end-to-side anastomosis; a great mobilization of the used biological graft occurs, damaging its inner layer, which generates the formation of thrombus; there is a big risk of kinking at the origin (angle of 90°) and risk of posterior wall perforation in the aorta at the moment the device is introduced under its light; the suture is substituted with disadvantages by stainless steel (9 pins, distant among them, maximizing the risk of bleeding).

Also as prior art, there is the CorLink Device, currently commercialized by Ethicon/Johnson & Johnson, that allows the creation of anastomosis between the ascending aorta and a saphenous vein segment. Aortic Anastomotic Device (AAD) is a self-expanded device with extra luminal nitinol constituted by a de um central cylinder with five interconnected elliptical arches and 2 groups of 5 pins in the end portion of the cylinder. The pins, after the eversion of venous walls in the device, fix the aggregate penetrating into the venous graft wall. A blade makes an opening in the wall of aorta and permits the coupling of AAD, which also fix the wall of aorta by pins. With this device: it poses a serious risk of bleeding, especially in friable aortas, thin, calcified or fibrous, restricting its applicability, also with risks, even in aortas with normal walls; in small gauge anastomosis, there is a risk of thrombosis, hyperplasia, intimal proliferation and fibrosis (reaction to foreign body type in origin of anastomosis) with consequent stenosis resulting in occlusion of anastomosis; sutures are used in some cases; there is cases of infarction caused by equipment; there is a recurring need of re-operations in patients; the device presents contact with blood flow (foreign body); it is not flexible; it does not multiple anastomosis; an inadequate mobilization of venous graft occurs, and can cause damage to its intimal layer, it could form thrombus; it is used only in extreme cases because it is a technique of complex usage; the suture is substituted by stainless steel in contact with blood flow.

Another known device is the St Jude Distal Connector that consists of a stainless steel clip mounted on a catheter, comprising a balloon for subsequent expansion and connector mounting. The catheter is introduced backward from the end, by doing a small hole in the anastomosis site, the clip fixes the vein in the hole, the catheter goes to coronary and releases the connector. The catheter is removed and a suture is done in side-to-side anastomosis. With St Jude Distal Connector, occurrence of leakage problems were detected in 20% of the used connectors; the use of a metallic clip requires due care for handling to avoid distortion in the anastomosis; late angiographies reveal smaller circular diameter of anastomosis made with o St. Jude Distal Connector, when compared to controls made with conventional suture; there is remarkably risk of bleeding and the graft is very mobilized, and lacerations can occur in its inner layer, allowing the formation of thrombus.

The HeartFlo™ is a multi-suture instrument for anastomosis with wires automatically applied in end-to-side and side-to-side anastomosis. The surgeon manually ties the suture wires (10 wires) and concludes the anastomosis similarly to the traditional process. Besides of being a product of complex handling, it makes suture in anastomosis (keeping the undesirable foreign body in the internal origin of the anastomosis) and is restricted to end-to-side and side-to-side anastomosis. There is also an excessive mobilization of graft, and can cause lesions in its intimal layer, which would be the inductor that forms the thrombus.

Another technique and known device is the Solem Graft connector, produced by the Swedish company Jomed. It is constituted by a stent made of nickel and titanium coated with polytetrafluorethylen used to connect the internal thoracic artery the left anterior descending coronary artery. The results has not been satisfactory, because it poses risk of bleeding; there is also an excessive mobilization of graft, probably damaging intimal layers, allowing the formation of thrombus; it is not flexible, by this fact, causes trauma to grafts; it does not make multiple anastomosis, at a single time; presents contact with blood flow (foreign body); and is frequent the need of-operations.

The Magnetic Vascular Positioner System is produced by Ventrica and comprises 4 magnetic rings and the anastomosis is processed by magnetic attraction of 4 ports. However, initial experimental results demonstrate leakage, also an undesired contact of materials with blood flow. On the other hand, it is necessary to be careful to avoid the capitation of excess of tissue among the magnets. With this system, there is also a need of suture in some cases; there is occurrence of infarction caused by equipment; and is frequent the need of-operations in patients; and also requires clamping.

Also, as a device known by the medical area, the Combined Anastomotic Device and Tissue Adhesive, developed by Grundeman & Borst group, combines micro mechanical technique with use of adhesive (glue). The use of this method can result in leakages and need traditional sutures; it is frequent the need of re-operation due to leakage/bleeding; and performs only one anastomosis at a time.

Finally, it is also experimentally practiced anastomosis assisted by laser, where the results are not different from conventional isolated sutures, because there is a need of suture in some cases; there is a risk of bleeding e leakage; and does not perform multiple anastomosis.

Even so divulged nowadays, anastomosis with clamper, by insecurity, and almost totality of surgeons perform conventional sutures throughout the route of anastomosis, with an intention of avoiding leakages and bleedings, it means the use of clampers just makes the procedure more expensive, once the conventional suture is also applied.

In short, the conventional anastomosis, with clamping and with suture, standardized in 1906 by Aléxis Carrel, remains the first choice for any type of anastomosis and organs to be anastomosed.

With an expectation of changing the current situation, the Brazilian patent no. Pi 9706197-2, describes and claims a prosthesis for vascular anastomosis, or in any other organ or tissue, without the use of clamping and sutureless, solving, in an elegant and efficient manner, the limitations inherent to prosthesis of the above mentioned prior art, when used in vascular anastomosis performed, mainly in thin aortas, calcified and friable; or in any other application where a clamping of a vein or artery can pose excessive trauma for conditions of a given patient. The prosthesis that is subject of that request allows the embodiment of fast and safe anastomosis, without obstruction of vein or artery lumen of which anastomosis is made, also allows anastomosis in tissues, veins or arteries in bad conditions and never would accept a clamping used in conventional anastomosis. This is achieved by a generally cylindrical shaped prosthesis with a flange orthogonally extending from its external side wall, in a point in the prosthesis length between its ends; the referred flange has openings distributed around its surface. The description of the usage method and specific construction of the prosthesis is presented in the drawings of the descriptive report of that request, as well as the document C19706197-2, Certificate of Addition of the first.

Although these anastomotic devices can be presented as suited to the purposes for which they were projected, they are not so suited for the purposes of the present invention, as described herein below.

### SUMMARY OF THE PRESENT INVENTION

The present invention refers to variations of anastomostic devices currently known in a way of enabling side-to-side, end-to-end and end-to-side anastomosis without clamping and sutureless wherein at least one vascular implant or any other it is inserted in the lumen of the prosthesis and it is reversed by jacketing for covering part of the prosthesis and it is affixed to the rigid part of the intraluminal part and/or to a flange. Prosthesis' tubular member may or not have intraluminal part and, as continuity, it may comprise metallic wires with elastic memory. Flange may or not have openings and chamfers allowing it to be sewed external to the tissue, vein, artery or any tubular organ in order to eliminate contact of strange bodies with the internal part of anastomosis, as well as the anastomosis to be done without clamping, which is the main responsible factor for tromboembolisms, friable wall lacerations and ischemia of organs due to clamping. It also permits wide anastomosis with no risk of implants elbowing, single or multiple, at a single time. The prosthesis may also have several dimensions and formats in order to accommodate several sizes and types of implants in a simultaneous way.

An objective of the present invention is to proportionate an anastomotic device having a tubular member comprising retractile shafts that will keep the implant open and related to the organ wall.

Another objective of the present invention is to proportionate an anastomotic device that much facilitates implant reversion.

Another objective of the present invention is to proportionate an anastomotic device that avoids protuberance in the set prosthesis-implant in the light of the organ and allows anastomosis without clamping and sutureless, without risk of implants elbowing, wide anastomosis, single or multiple in a single time, of any kind between any of the two tubulars.

Additional objectives of the present invention and other modalities will appear as the depiction proceeds. Such modalities will be depicted in enough details in order to make it possible technicians in the art to implement the invention. Besides, it is to be understood that other modalities may be used, and structural changes may be done without withdrawing from this invention scope. In the attached drawings, symbols of similar reference designate the same parts or similar ones for all several views.

Thus, the next depiction detailed is not to be taken in a restricting sense and the scope of the present invention is better defined by the attached claims.

### BRIEF DESCRIPTION OF THE DRAWINGS

In order the invention may be better understood, it will be now depicted, by means of example, with reference to the attached drawings, which:

FIG. 1 illustrates a perspective view of a first concretization of the prosthesis with intraluminal part having a rigid part, in a proximal and distal way, metallic wires with elastic memory, comprising holes, double holes or external handles for fixing the implant to the rigid part of the intraluminal part and/or to the flange.

FIG. 1A illustrates another concretization of prosthesis that differs from FIG. 1 prosthesis because the shafts are not fixed to the tubular member, they go through holes it has, and they are long, prolonging up and down from the flange.

FIG. 2A illustrates a perspective view of another concretization of the prosthesis composed of two parts. One with intraluminal part having a solid internal flange and tubular member having small rigid part (wherein implants can be fixed with a circular stitch) prolonged upwards and outwards by metallic wires with elastic memory. Internal flange may comprise holes, double holes or external handles for fixing the implant to the flange, if preferred, and/or for fixing prosthesis to the organ's wall. The second part refers to a external flange which may also have holes, double holes or small handles separated from the first part, that when coupled promote sandwiching of the vase or any other organ wall.

FIG. 2B illustrates a view of the prosthesis illustrated on FIG. 2A, in use in a vascular anastomosis.

FIG. 3A illustrates the view of another concretization of the prosthesis with tubular member having flexible metallic wires and elastic memory, metallic wires being able to articulate, oscillating from 0 to 900 in relation to tubular member similar to a second flange.

FIG. 3B illustrates a cross-sectional cut view of FIG. 3A making evident a way of articulation of a wire that makes the intraluminal flange.

FIG. 4A illustrates a perspective view of another concretization of the prosthesis having two parts of flexible wires from which one has elastic memory, intraluminal part and the other does not (it maintains deformation), the external part, opposed and fixed to a rigid and compact central part, metallic wires being similar to two flanges.

FIG. 4B illustrates another concretization of the prosthesis in which the handles are not fixed to the rigid ring, going through existent holes in such ring. In fact, this prosthesis differs from FIG. 1A prosthesis because the rigid part of its tubular member has no flange. The reason of the mobile handles is the same: making it easier reversion of implants and guaranteeing complete absence of protuberances on the prosthesis-implant set in the light of the organ.

FIG. 4C illustrates a perspective view of another concretization of the prosthesis having two parts of wires, metallic or not, opposed, interconnected in their center by a ring, rigid or flexible, having elastic memory or not, formed just by a wire. External part of the ring is flexible and without elastic memory maintaining the deformation suffered. Intraluminal part, internal to ring, has elastic memory and recovers the form after deformation.

FIG. 4D illustrates a prosthesis that differs from prosthesis from FIG. 4C because its handles are not fixed to the ring, going through holes it has, allowing the ring to go up and down.

FIG. 4E illustrates a superior external view of the concretization illustrated on FIGS. 4, 4B, 4C, and 4D after being folded by wires, whether metallic or not, in order to settle them to surface superior of the organ wall.

FIGS. 5A-5N illustrate different forms and numbers of articulate intraluminal flanges.

FIGS. 6A-6C illustrate different forms of flanges formed by metallic wires.

FIG. 7A illustrates a perspective view of another concretization of the prosthesis having intraluminal part that has metallic wires with elastic memory and external flange in spring format of spiraled wires.

FIG. 7B is a superior view of the spiraled flange illustrated on FIG. 7A.

FIG. 7C illustrates a perspective view of a concretization of the prosthesis in at least two parts which, when summed up, close the tubular body circumference.

### DESCRIPTION OF THE PREFERRED MODALITIES

Referring now to the drawings in which characters of similar reference denote similar elements for all several views, the figures illustrate one of the embodiments of the present invention in a prosthesis format for anastomosis having at least one intraluminal part and at least one flange comprised of metallic wires or any other, having elastic memory or not.

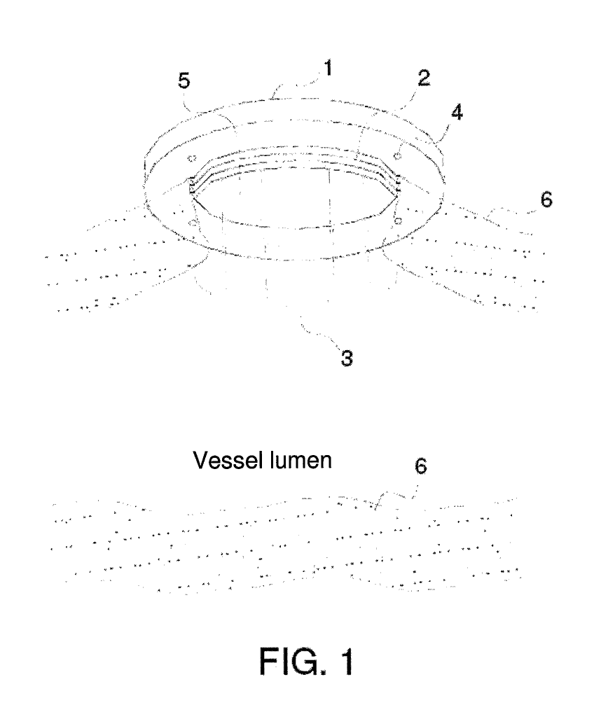

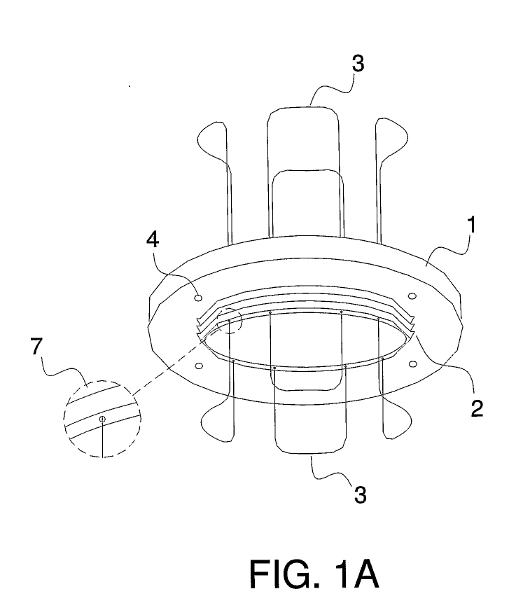

FIG. 1 illustrates a prosthesis with external flange 1 having holes 4, double holes or small handles (non presented), and tubular member comprised of two parts: the first one rigid 2, compact having external grooves 5 wherein implants can be fixed by circular stitches after being everted, and the second part, continuing the rigid part, comprised by metallic wires 3 or any other flexible biocompatible material and having elastic memory that back to its original form and position after deforming, continuous or discontinuous with a variable number of shafts 3 (from wires). Such shafts 3 due to being flexible make the procedure of implant eversion very fast and simple, making the implant to be fixed in the rigid part 2 with circumferential stitch or even on the holes 4 double holes and/or flange handles. A technical concretization for its application could be depicted as: pass the implant, of any type and gauge compatible with prosthesis light, through its light, evert it, compress the shafts 3 in order to diminish diameter, recover with the everted segment of the implant that is, then, fixed with circumferential stitch in the rigid part 2 of the intraluminal part, over external grooves 5 that it has; the stitches are applied through holes 4 of flange 1 following the track: from up to down in the holes 4 of flange 1 (wires handles will be supported on the edges that stay above the tubular member—not presented—or fixed in the double holes or existent handles between the represented holes 4, so that they do not interfere to the prosthesis light), up to down, transfixing or not, on the organ wall that will receive the anastomotic set (prosthesis-implant), adjacent to the local of incision that if it is rectilinear it will be the extension of half perimeter of the external diameter of the prosthesis' tubular member. After wires being passed, two or four, two by two opposed and having parallel legs, the incision is made, it is digitally covered, the anastomotic set is inserted and wires are tide. Anastomosis is then performed without clamping and sutureless, and several implants can be used with a single prosthesis since they are previously linked in an anastomotic trunk, performing, thus, a wide, multiple proximal anastomosis in a single time without clamping and sutureless. In this anastomosis there is no elbowing in the implants, regardless where they are being positioned or which pressure they are subjected to. It also refers to a hermetically closed anastomosis once the organ wall is compressed against intraluminal part and under prosthesis' flange 1 guaranteeing even more hermetic character of such anastomosis. FIG. 1A illustrates an important variation of prosthesis from FIG. 1 wherein metallic wires 3 or any other are not fixed to tubular member who contains rigid part 2 and flange 1. They go through holes 7 and they can be tracked up or pushed down before or after anastomosis to be done aiming at facilitating even more the implant eversion and its fixation to the rigid part 2 from tubular member, and also, and mainly, retracting the wires, after the anastomosis to be done in order to annul any protuberance in the anastomotic set in the light of the organ. These wires 3 may have elastic memory or not. If they do not have it, they need to be resistant enough so that they are no easily deformed.

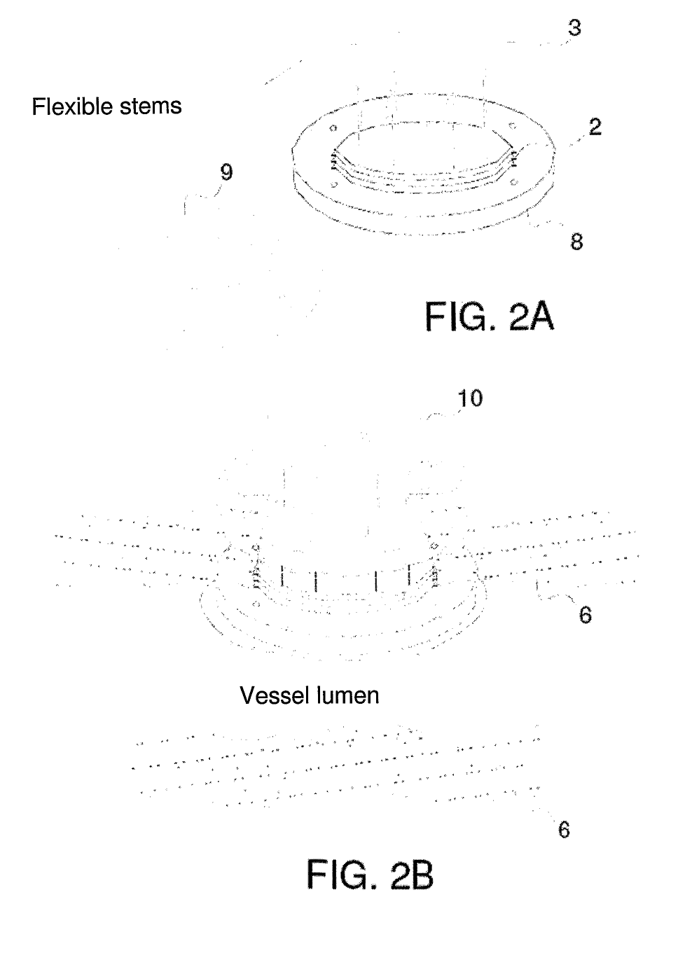

FIG. 2A illustrates another concretization of the prosthesis comprised of two parts: the external part 9 that is just a flange 9 having holes 4, double holes or small handles, and the intraluminal part that may be exactly the same the FIG. 1, but to upside down. Thus, it will have an intraluminal flange 8 having holes 4, double holes or handles and also a rigid segment 2, having external grooves 5 wherein implants can be fixed, which continues upwards with metallic wires 3 or any other biocompatible material, whether having elastic memory or not. In this case, in order the intraluminal flange 8 can be covered without harming the natural diameter of the implant or implants used, it must be widen, if only one, or united in a anastomotic trunk if multiple, which caliber is the same or fairly higher than external diameter of intraluminal flange 8. External flange 9 may be jointed to metallic wires 3, before or after the introduction of the anastomotic set. The more metallic wires 3 are tracked outside through stitches between the metallic handles 3 and the organ wall, the more there will be sandwiching of the organ wall, homeostasis and certainty of annulling any protuberance on the anastomotic set in the light of the organ. These stitches may be unnecessary, by only folding with a clamp the handles 3 without elastic memory over external flange 9. The technique previously depicted will be able to equally be herein applied. Several technical concretizations for application of such prosthesis are possible. For example, application of only one stitch that goes through external flange 9 and performs a suture in a pouch on the organ wall circulating the incision local. After incision and introduction of anastomotic set, the suture in pouch is tide.

FIG. 2B illustrates a view of the prosthesis illustrated on FIG. 2A, in use in a vascular anastomosis.

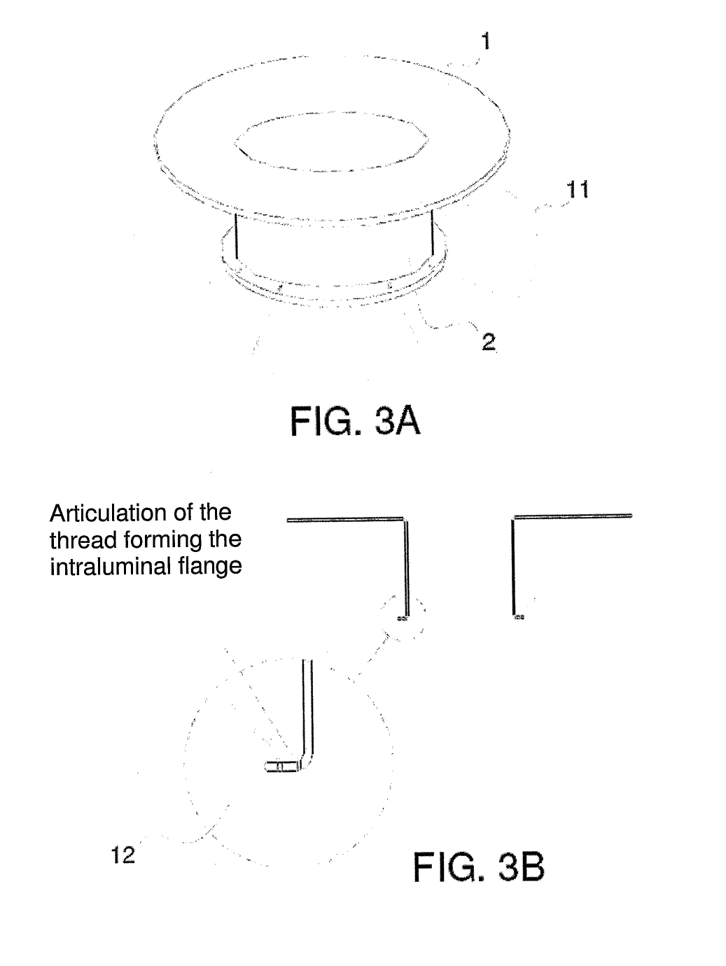

FIG. 3A illustrates a prosthesis having a rigid external, or flexible in tissue, flange 1, with or without holes 4, double holes or small external handles having no rigid tubular member 2 that has external grooves 5 for fixing implants (not presented), and externally to its distal end, metallic handles 3, or made of any other biocompatible material whether having elastic memory or not, that oscillates from 0 to 90 degrees in relation to the tubular member 2. These tilting handles intend to make it easier the implant eversion for covering the intraluminal part of the tubular member 2, and also avoid non-protuberance of the implant prosthesis set in the light of the organ.

FIG. 3B illustrates a view in cross-sectional cut on FIG. 3A making evident a possible form of articulation 12 of the wire that forms the intraluminal flange 11, that guarantees a maximum oscillation of 90 degrees in relation to the tubular member 2.

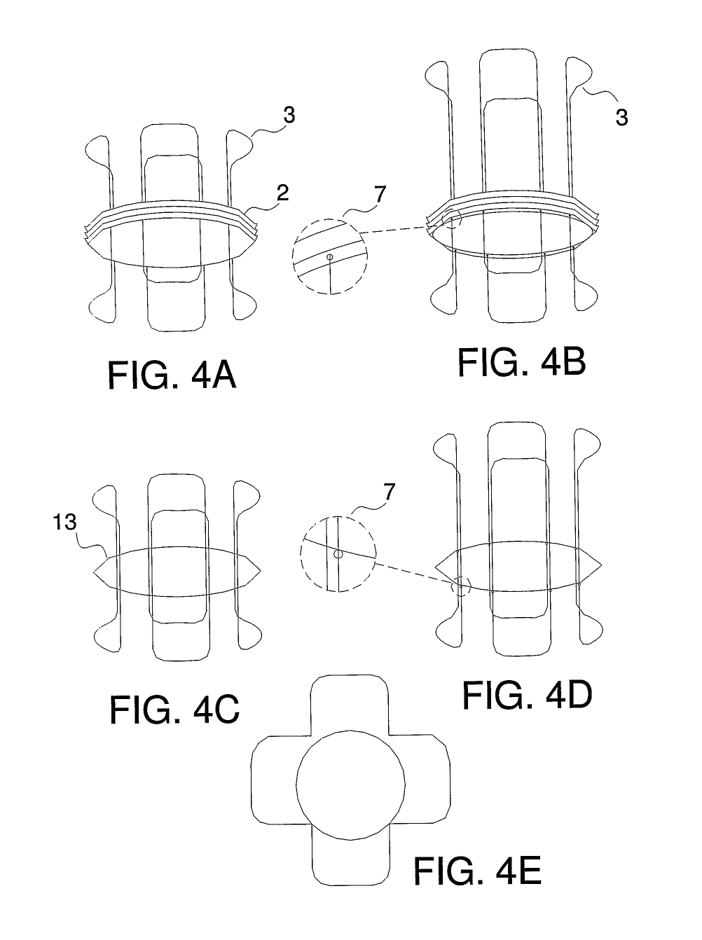

FIG. 4A illustrates another concretization of the prosthesis having two parts of metallic wires 3 or any other biocompatible material, flexible or rigid, whether having elastic memory or not, interposed, and fixed to it through rigid part 2 having external grooves 5 for fixing the implants. The number of handles 3 is variable depending on the characteristics of the organ wall that will receive it: the more friable, calcified or fibrosed, the higher the amount of handles 3 in order to contain the possible fragments that may interpose to the anastomosis origin. It might have handles 3 with same or different characteristics. If the same, any of them can be the external or intraluminal ones. If different, it could be like that: the external one, having no elastic memory, should accept and maintain the suffered deformation, and the internal or intraluminal ones, having elastic memory, recovering its original form after deformation. A simple and fast way of applying it could be depicted like this: make an incision on the organ wall in the center of a suture in pouch, an anastomotic set is inserted and the suture in pouch is smoothly tracked; the external handles are folded and fixed to the organ wall with single stitches; the suture in pouch tiding is completed. Anastomosis made without clamping and sutureless, being single or multiple at one time.

FIG. 4B illustrates another concretization of the prosthesis in which handles 3 are not fixed to the rigid ring 2, going through holes 7 existents in this ring. This prosthesis in fact differs from FIG. 1A prosthesis due to the fact that the rigid part of its tubular member has no flange. The reason of the mobile handles 3 is the same: make it easier the eversion of implants and guarantee the complete absence of protuberance of implant prosthesis set in the light of the organ.

FIG. 4C illustrates another concretization of the prosthesis that differs from FIG. 4A prosthesis only for its ring 2 being composed by only one wire 3, that can be flexible or rigid, whether having elastic memory or not. This ring 2 is fixed to handles 3.

FIG. 4D illustrates another concretization of the prosthesis that differs from FIG. 4C prosthesis only for its ring 2 not being fixed to handles 3 and having holes 7 wherein they pass through, being able to vary its position throughout them.

FIG. 4E illustrates the superior aspect of the final prosthesis presented on FIGS. 4A-4D after their external handles are folded, settled and fixed on the superior surface on the organ wall that received them.

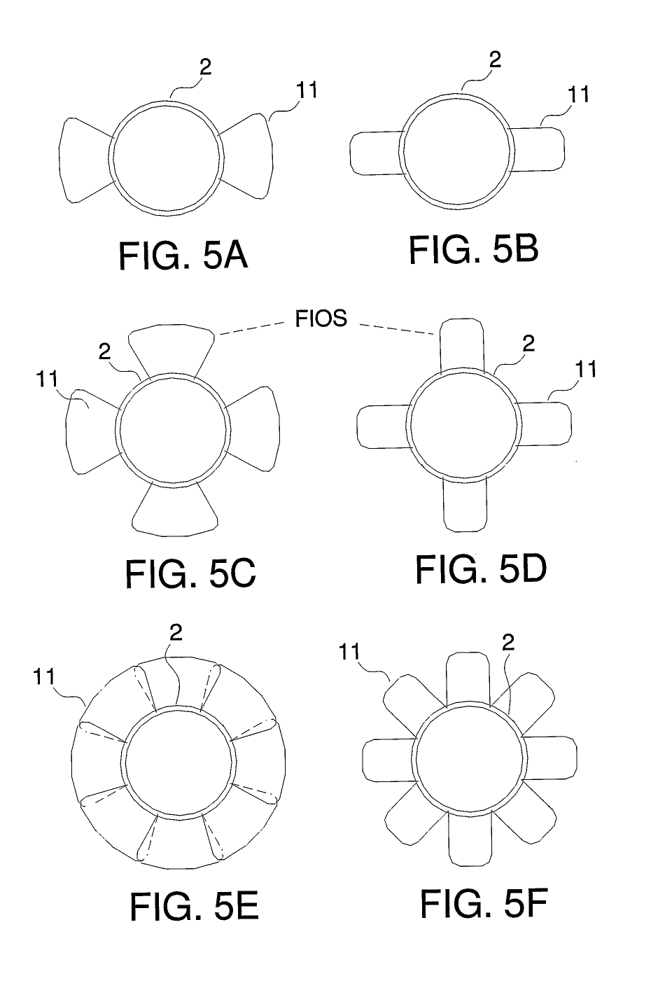

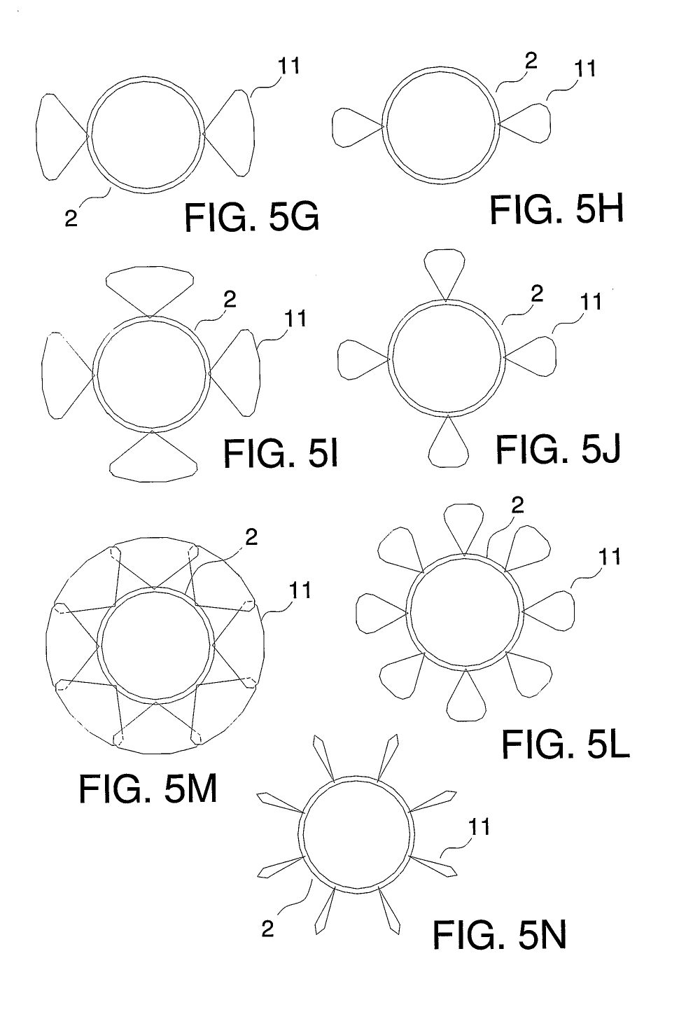

FIGS. 5A-5N illustrate different forms and numbers of articulated intraluminal flanges 11, tilting, whether formed by metallic wires 3 or not, flexible or not, with elastic memory or not, all intending to make it easier the eversion of the implants, and avoid protuberance of implant prosthesis set in the light of the organ.

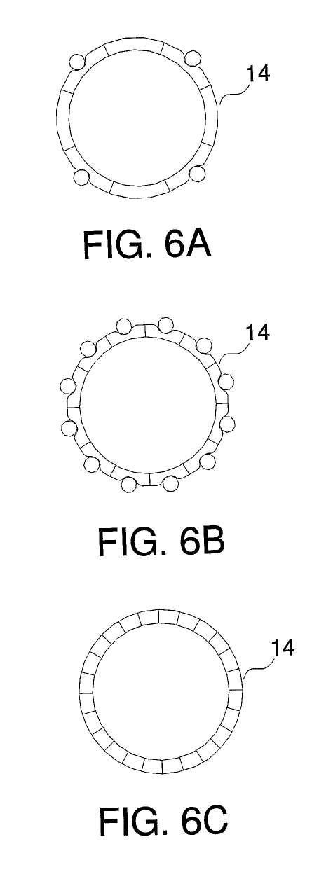

FIGS. 6A-6C illustrate different forms of external flanges 14 whether formed by metallic wires 3 or not, flexible or not, with elastic memory or not, intending to make it easier the application of prosthesis fixing wires to the organ wall, to avoid interposition of wires handles to prosthesis light, besides being able also to be the fixation local of prosthesis implants. The higher the amount of handles or holes, the higher this versatility will be.

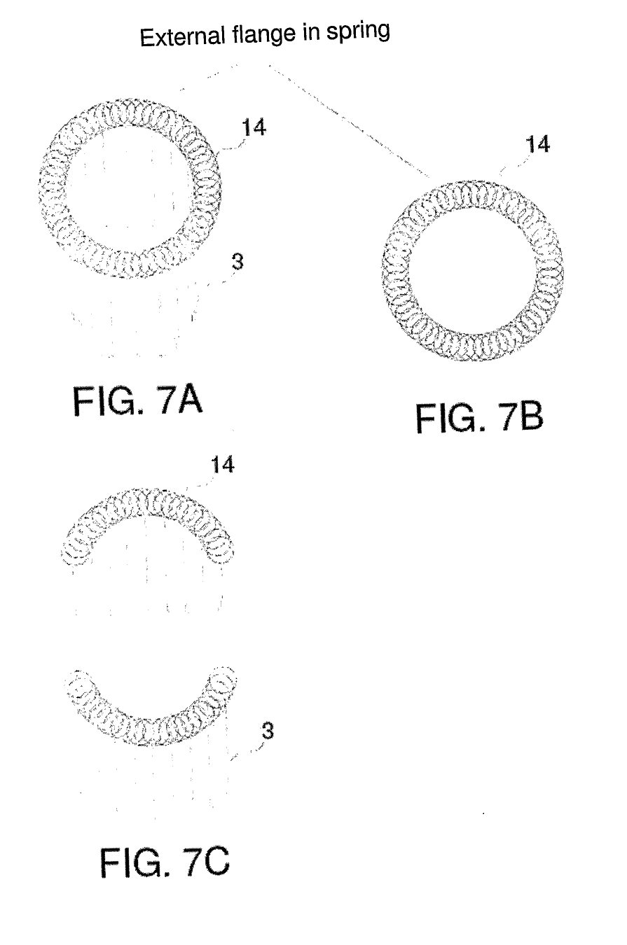

FIG. 7A illustrates a perspective view of another concretization of the prosthesis with intraluminal part whether formed by metallic wires 3 or not, flexible or not, with elastic memory or not, in a variable number, and the external flange 14 in a spring or spiraled wires format, also flexible or not and having elastic memory or not. If handles 3 and flange 14 are flexible and have elastic memory, it will be easier the implant eversion to cover the intraluminal part as well as their fixation to prosthesis, besides great facility, due to prosthesis malleability, of application of the prosthesis fixation wires, as well as suiting the prosthesis to the organ wall surface.

FIG. 7B is a superior view from spiraled flange 14 of FIG. 7A.

FIG. 7C illustrates a perspective view of a concretization of the prosthesis in at least two parts, that when summed up, they close the circumference of the tubular body. This prosthesis besides making it easier the implant eversion, it also can be partially used and also in video surgeries, since it can go through trocarters that have caliber equal to each of their halves.

1. Prosthesis for anastomosis comprising: at least one tubular member that comprises resistant wires (3).

2. Prosthesis, according to claim 1, wherein the resistant wires (3) are biocompatible, and are at least one of: metallic, non-metallic, flexible, non-flexible, or characterized as having elastic memory, and form internal or external handles.

3. Prosthesis, according to claim 1, wherein the resistant wires (3) are elongation fixed of the tubular body (2).

4. Prosthesis for anastomosis comprising: at least one rigid tubular member (2), that is compact, ring-like, wherein it is not fixed to handles wires, and comprises holes (7) through which metallic handles may go through or not.

5. Prosthesis, according to claim 4, wherein the ring (13) is composed of a single wire.

6. Prosthesis, according to claim 1, wherein the resistant wires (3) are articulated (12) to the tubular body (2).

7. Prosthesis, according to claim 1, wherein resistant wires (3) form at least one flange (11).

8. Prosthesis, according to claim 1, wherein resistant wires (3) form at least one flange (11) and at least one intraluminal part.

9. Prosthesis, according to claim 1, wherein resistant wires (3) form at least one spiraled flange (14).

10. Prosthesis for anastomosis comprising: at least two parts, wherein resistant wires (3) form at least one intraluminal part having an end fixed to a rigid flange (2), the prosthesis also comprising another flange (9) that is solid and external, to be sandwiched against the organ wall by joint, by lumen, to wires, externally.
